(19) United States
(12) Patent Application Publication
(10) Pub. No.: US 2026/0238351 A1
(43) Pub. Date: Aug. 13, 2026

(54) DATA TRANSMISSION SYSTEM FOR COMPUTER TOMOGRAPHS WITH A WAVEGUIDE

(71) Applicant: Venturetec Rotating Systems GmbH, Kaufbeuren (DE)

(72) Inventors: Harry Schilling, Schwabach (DE); Philipp Robi, Jengen (DE); Grigorii Bubnov, Kaufbeuren (RU); Stephan Neubauer, Forchheim (DE)

(21) Appl. No.: 19/180,812

(22) Filed: Apr. 16, 2025

(30) Foreign Application Priority Data
Aug. 2, 2024 (DE) 102024002510.3
Mar. 27, 2025 (EP) 25166764.8

(51) Int. Cl. H04B 10/80; G02B 6/12

(52) U.S. Cl. CPC H04B 10/801; G02B 6/12009

(57) ABSTRACT

A device according to the invention for transmitting data between the rotating part and the stationary part of a computer tomograph with a waveguide slotted in the longitudinal axis for transmitting high-bit-rate data signals

The invention relates to a data transmission system for transmitting data between two parts which can rotate relative to one another about a common axis, for example a rotating part and a stationary part of a rotary transmitter, such as in a computer tomograph, by means of a split waveguide or hollow conductor.

A device for data transmission in computer tomographs is known from U.S. Pat. No. 6,433,631. A transmitter signal is applied to a strip line in the rotating part. A tap is provided on a stationary part, which is routed at a small distance in the order of approx. 1 mm from the stripline.

The transmission systems known from the state of the art are limited to data rates of max. 10 GBit/s.

Further prior art is known from DE 35 38 035 A1 and DE 32 09 906 A1.

Other devices based on traditional waveguide systems used to transmit high-frequency signals comprise significant limitations, especially when used in rotating systems. Previous solutions such as slotted waveguides offer limited bandwidth and comprise high insertion loss, which impairs transmission quality.

The purpose of the invention is to present a data transmission system which permits data transmission rates of up to several 100 GBit/s and can be integrated into computer tomographs, for example, with little mechanical effort.

A solution to this task according to the invention is given in the independent claims. Further embodiments of the invention are the subject matter of the dependent claims.

The invention is described below by way of example embodiments with reference to the drawings.

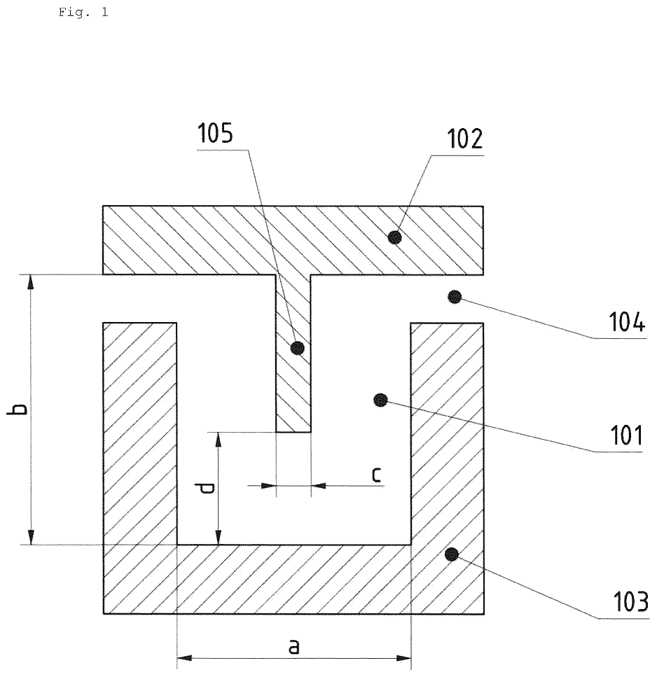

FIG. 1 schematically shows a waveguide arrangement with a stator and a rotor with a rib according to a first embodiment of the invention.

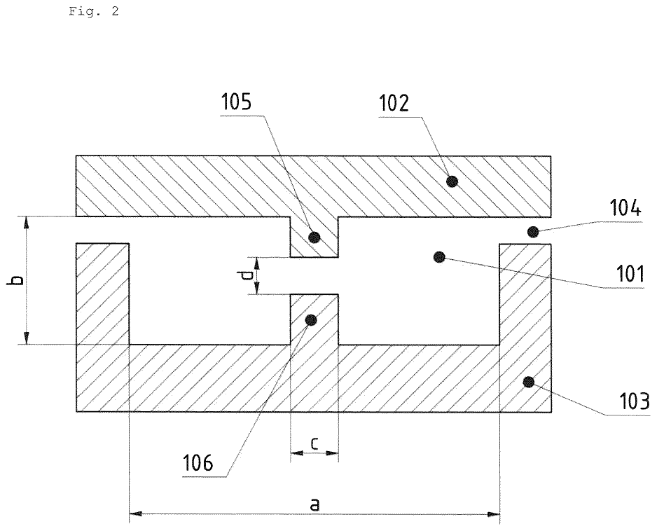

FIG. 2 schematically shows a waveguide arrangement with a stator and a rotor with a rib according to a second embodiment of the invention.

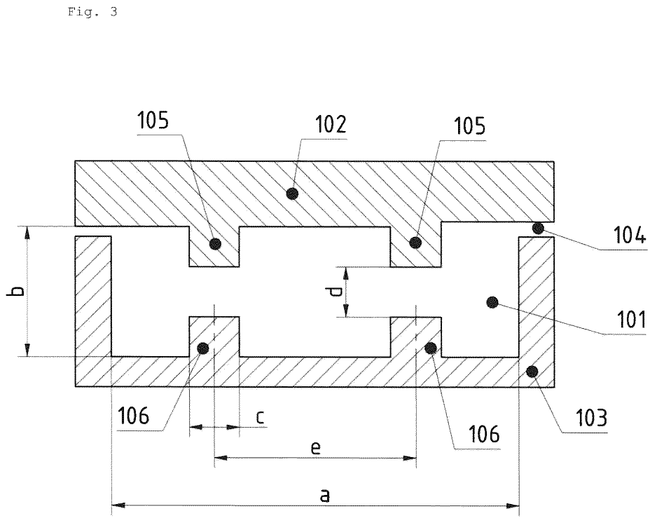

FIG. 3 schematically shows a waveguide arrangement with a stator and a rotor with several ribs according to a third embodiment of the invention.

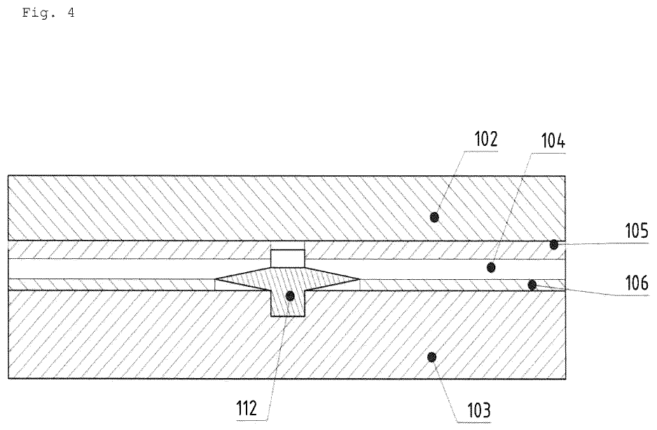

FIG. 4 schematically shows a waveguide arrangement with a stator and a rotor with an absorber for terminating the RF signal.

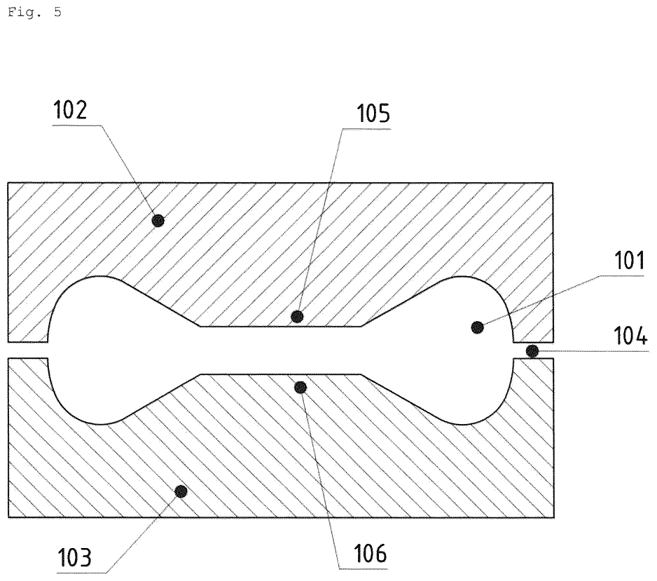

FIG. 5 schematically shows a waveguide arrangement with a stator and a rotor with a rib and a bone-shaped waveguide cross-section according to a further embodiment of the invention.

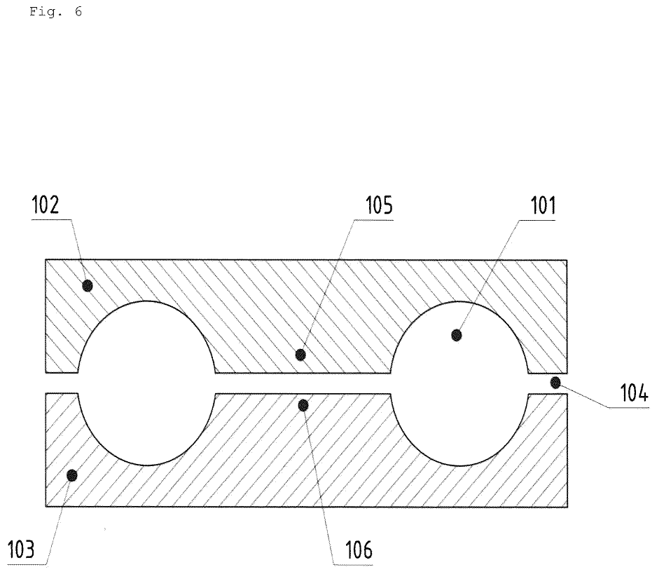

FIG. 6 schematically shows a waveguide arrangement with a stator and a rotor with a rib and a circular waveguide cross-section according to a further embodiment of the invention.

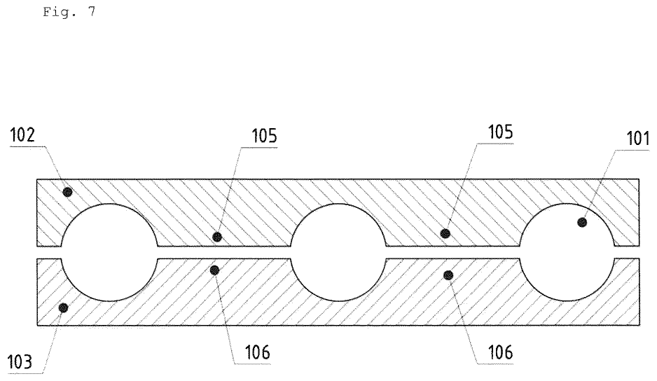

FIG. 7 schematically shows a waveguide arrangement with a stator and a rotor with several ribs and a circular waveguide cross-section according to a further embodiment of the invention.

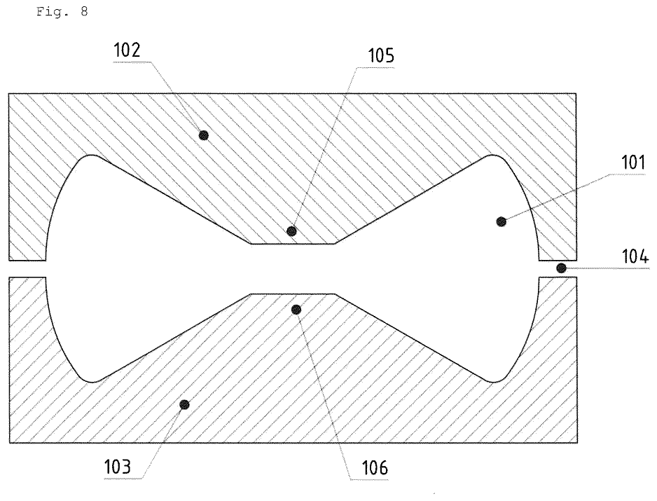

FIG. 8 schematically shows a waveguide arrangement with a stator and a rotor with a rib and a trapezoidal, rounded waveguide cross-section according to a further embodiment of the invention.

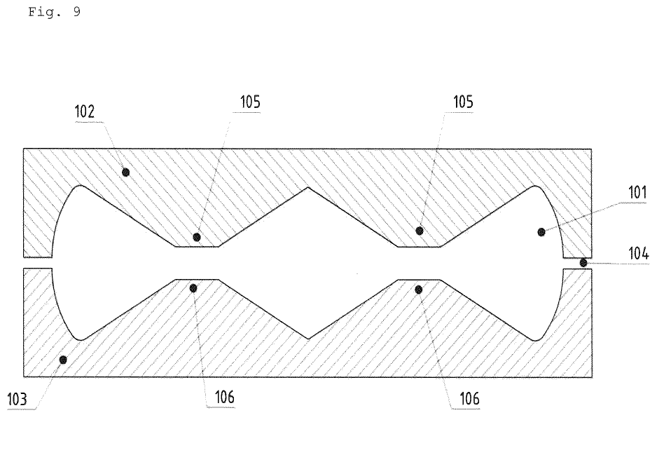

FIG. 9 schematically shows a waveguide arrangement with a stator and a rotor with several ribs and a trapezoidal, rounded waveguide cross-section according to a further embodiment of the invention.

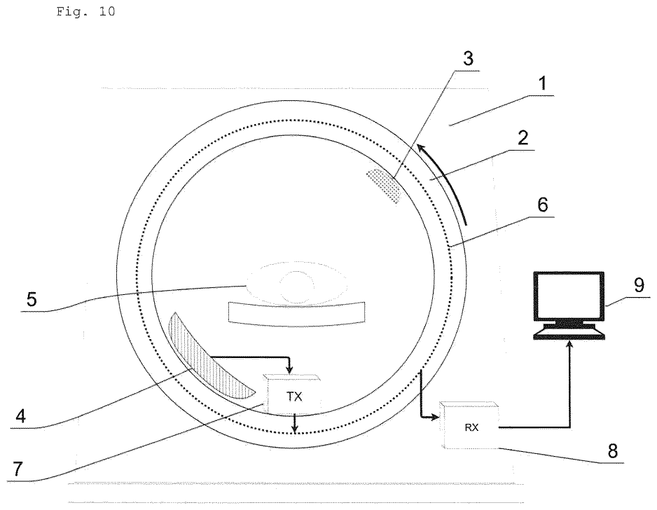

FIG. 10 schematically shows an application of the waveguide transmission system in a computer tomograph.

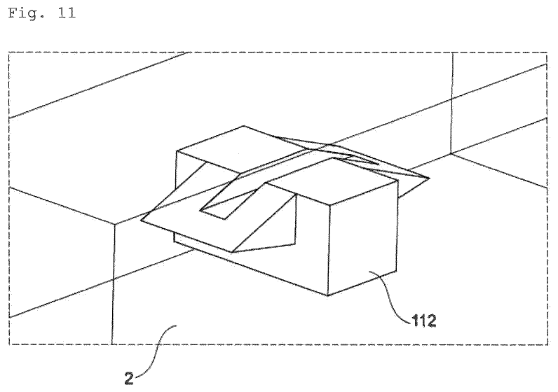

FIG. 11 shows a detailed example of the configuration of the termination of a waveguide according to the invention.

In the following, the invention is described in detail using the example of a computer tomograph, but is not limited to this.

A device according to the invention for transmitting data between a rotating part or rotor 102 and a stationary part or stator 103 of a computer tomograph comprises, for example, a data source on the rotating part and at least one data sink on the stationary part. A data source can be, for example, an X-ray detector or the data acquisition system (data processing system) or a control device or a computer. A data sink can be a computer for evaluating and processing the data, but also another control unit or an FPGA, etc.

The invention is not limited to use with a computer tomograph. It is also not necessary that the stator 103 is actually and always stationary. It is sufficient that the two rotating parts can rotate relative to each other about a common axis. Also, the transmission of data is not limited to the direction from the rotor 102 to the stator 103. Transmission in the opposite direction and bidirectional transmission are also possible.

Furthermore, at least one transmitting device or transmitter 7 and a first waveguide 101 fed by it, here in the form of a waveguide running in a ring around the common axis, with a specific structure and optimized dimensions is used in the rotor 102. This waveguide 101 enables low-loss transmission of high-frequency signals and reduces the dispersion effects caused by rotation.

A waveguide is a conductive, usually metallic tube that conducts high-frequency electromagnetic waves, typically in the frequency range from 1 to 1500 GHz. It comprises a cavity that is separated from the outside by conductive walls and contains no internal conductors. Electromagnetic waves propagate inside the waveguide by reflecting off the metallic walls. The waves propagate in a single mode within the waveguide, with the electric field (E field) and the magnetic field (H field) being perpendicular to each other and oscillating orthogonally to the longitudinal axis of the waveguide. An electric field forms in the waveguide in the center of the wider side (a), which decreases towards the narrower sides (b). The magnetic field is created by the electric field and cannot be perpendicular to the metallic wall. The fields change their intensity and polarity to the rhythm of the input signal. The wave propagation in the waveguide is frequency-dependent. There is a so-called cut-off frequency below which no wave propagation takes place. This frequency depends on the dimensions of the waveguide, in particular the width (a). The wavelength of the wave to be transmitted must be smaller than the cut-off wavelength for propagation to be possible. Various modes (wave types) can propagate in the waveguide, which are referred to as Hmn or Emn waves. These modes arise from the solution of Maxwell's equations under the boundary conditions of the waveguide. The fundamental wave in a rectangular waveguide is the H10 wave, which is stable over a large frequency range when b/a≈0.5 is selected. Waveguides are used in ultra-high frequency and microwave technology as they comprise lower losses compared to coaxial cables and can transmit high power without causing significant losses or voltage breakdowns. In summary, a waveguide enables the almost lossless transmission of short electromagnetic waves, whereby the propagation is determined by the geometry of the waveguide and the modes used.

The waveguide 101 is designed to allow mechanical movement between the rotor 102 and stator 103 while efficiently transmitting electromagnetic power. The transmitter 7 receives data from the data source, such as the X-ray tube 3, and converts it into a corresponding electromagnetic signal for transmission or coupling into the waveguide 101. Furthermore, at least one receiving device or receiver 8 is provided in the stator 103, which receives or decouples the signal from the waveguide 101. The receiver 8 converts the signals for forwarding to the data sink, e.g. a computer 9.

The signal is transmitted from the rotor 102 to the stator 103 by means of a signal line in the waveguide 101.

The signals can be modulated and/or coded by the transmitter 7.

To avoid multipath propagation, mode selective feeding of the signal can ensure that the transmitted signal remains clear and consistent. This technique also minimizes the effects of the Doppler effect caused by the rotation of the rotary transmitter and improves the signal quality.

A mode selective feed is achieved by optimized T-waveguide connections, whereby the dimensions are optimized for a better standing wave ratio (VSWR).

To minimize the channel impulse response and avoid multipath propagation, a single-mode wave is preferred. Multi-mode propagation would lead to a high respectively wide impulse response due to different propagation speeds.

In the invention, the waveguide 101 is slotted or divided along the longitudinal axis. Both parts of the waveguide 101 resulting from the slitting are arranged with a gap 6, 106 at a small distance from each other, movable about the central axis of rotation. This is referred to as a waveguide system or waveguide system.

Based on the necessary transmission properties, the waveguide 101 may comprise a round, butterfly-shaped, T-shaped or double-T-shaped or angular cross-section, as shown in the various embodiments of the invention in the figures, with the angular cross-section being preferred. The profiles can also deviate from the basic shape by adding rounding or tapers.

In order to minimize leakage losses at the slots of the waveguide 101, single or multiple ribs 105 are introduced in the cross-section of the waveguide 101, which concentrate the electromagnetic fields and reduce the insertion loss. The web structure optimizes the mode propagation and minimizes the losses due to the focused concentration of the electromagnetic field in the center of the waveguide.

The ribs 105 can be designed as a single rib, double rib or quadruple rib. The shape and dimensions of the ribs can be optimized to reduce insertion loss and suppress undesirable modes.

One configuration of the invention provides at least one transmitter 7 in the stator 103 and at least one receiver 8 in the rotor 102. This configuration also enables communication from the stator 103 to the rotor 102.

A further configuration of the invention provides for a plurality of transmitters 7 and a plurality of receivers 8, each of which is arranged either on one of a plurality of waveguides 101 arranged in parallel or on a waveguide 101 subdivided into a plurality of circular segments. In the latter case, the number of receivers 8 is not necessarily but preferably n+1 and the number of transmitters 7 is n.

Termination 112 of the waveguide 101 may be necessary for certain applications. This depends on the configuration and number of transmitters 7 and receivers 8. Termination 112 is provided by pyramid-shaped microwave absorber material positioned in the waveguide 101.

A further configuration of the invention provides a waveguide 101 which transmits one or more signals from the rotor 102 to the stator 103 as well as from the stator 103 to the rotor 102. The signals can use different carrier frequencies and/or be differently modulated and/or differently coded.

The modulation can, for example, be an amplitude or frequency modulation or a mixture of both, such as QAM.

In the case of simultaneous transmission of several signals in a waveguide 101, a signal/frequency crossover or similar must be provided in both the transmitter 7 and the receiver 8, which ensures decoupling between the signals and transmission directions by means of suitable measures such as frequency selection, phase selection or direction selection.

In a further configuration of the invention, the transmitted signal is adjusted based on one or more selection parameters. The selection parameters can be, for example, the signal strength, the signal quality such as noise, amplitude, error vector magnitude, etc.

In another configuration, the invention is implemented as an array. Such an array comprises several waveguides 101, which are fed with signals that are in a defined relationship to one another in order to obtain a specific radiation pattern as a whole. Such an array can be designed with fixed phase relationships between the individual radiators or also with variable phase relationships.

In one configuration, a control unit is provided which, if given, sets or selects individual waveguide segments and the associated transmitters 7 and receivers 8 according to predetermined parameters. The predetermined parameters for setting or selection are, for example, signal level, signal-to-noise ratio, bit error rate, propagation time and/or phase shift in relation to a reference signal or a position signal.

The waveguides 101 are constructed from conductive material, such as metal, or from a carrier material coated with conductive material, e.g. plastic. If coated substrate material is used, it may be useful to slit your waveguide 101 laterally so that the coating material can be distributed in the waveguide 101 and adhere to the substrate material. Preferably, the coating in the waveguide 101 is applied galvanically or chemically.

A further configuration of the invention provides that an additional control unit is provided for controlling bidirectional communication based on time windows, which specifies the time frame for each communication direction.

According to a further configuration of the invention, at least one circuit for frame and/or data recovery is provided.

A further configuration of the invention comprises a signal processor or FPGA in the transmitter 7, which divides the data into several waveguides 101 or waveguide segments, and an electronic circuit, e.g. also based on a signal processor or FPGA, in the receiver unit, which recombines the data into a data stream.

A further configuration of the invention comprises an amplifier directly at the signal coupling-in point (feed-in point) and/or at the signal coupling-out point (receiving point) upstream of the receiver 8, wherein the gain of the amplifier is variable and the gain is adjusted on the basis of measured or predetermined parameters.

A further configuration of the invention comprises a discrete or integrated evaluation circuit which, on the basis of various quality criteria, such as the error rate, selects the receiving unit which best fulfills the quality criteria for forwarding the signal in the case of several receiving units.

To simplify the illustration, reference is made in this document to a transmission from the rotor 102 to the stator 103 of a computer tomograph. Of course, a device according to the invention can also be used in the opposite direction of transmission. Similarly, a device according to the invention can also be used in other applications for rotary transmission and also for linear transmission of two units moving relative to each other.

The direction of transmission according to the invention was chosen from the rotor 102 to the stator 103, as this corresponds to the most common application. However, transmission in the opposite direction or bidirectionally is also possible.

The invention presents a novel principle for transmitting broadband microwave signals between the rotor 102 and stator 103 of a rotary transmitter. A key feature of this approach is the ability to transmit single broadband waves comprising lower channel impulse responses compared to multimode waves. This special feature considerably simplifies and expands the possibilities of data transmission.

The data transmission system of the invention is designed to meet the requirements associated in particular with future photon counting CT scanner applications.

This includes a targeted high data rate of 40 Gbit/s, preferably up to 270 Gbit/s. Compared to known approaches, such as capacitive methods (which are limited to 10 Gbit/s per second), significantly higher data rates can be achieved.

For example, 65 Gbit/s are transmitted via a single channel, based on a spectral data density of 3 bits/second/Hz and a standard V-band bandwidth of 25 GHz (including guard intervals). The V-band is a frequency range in the microwave spectrum that extends from 50 to 75 GHz. It is used as a standard designation by the IEEE (Institute of Electrical and Electronics Engineers).

To achieve a data rate of more than 40 Gbit/s, a bandwidth of more than 15 GHz is required, depending on the modulation scheme selected.

Consequently, the system must comprise a relatively flat amplitude ratio.

According to the frequency regulation laws of the ITU (International Telecommunication Union) and the special propagation characteristics of radio waves in the atmosphere, only frequencies above 51.4 GHz can be used without special permission to achieve a bandwidth of 15 GHz or more.

Therefore, the V-band and the E-band, which extends from 60 to 90 GHz, are a viable option.

The invention is based on principles applicable to structures for any band, taking into account the scalability of passive components, but limited by mechanical manufacturing tolerances. For the sake of clarity, the concept according to the invention is considered for the V-band in the following discussion.

The system must comprise a relatively low specific insertion loss per unit length over the specified bandwidth. A loss value of more than 40 dB/m is considered high.

It may then be necessary to increase the range by using AGC (automatic gain control) for the transmitter-to-receiver system.

Since the invention is based on a waveguide 101, it is critical to utilize a single mode wave within the waveguide 101 and minimize mode dispersion as much as possible. This ensures that the impulse response of the channel remains as short as possible. If a multi-mode wave were used, this would result in a long impulse response with multiple echoes due to the different propagation speeds (i.e. dispersion) of the different modes and echoes.

In CT applications, the data transmission system must be arranged around the circumference of the rotary transmitter with a diameter of more than 1.2 m. In the CT configuration, the transmitter 7 and receiver 8 rotate against each other at a predetermined speed, for example up to 300 revolutions per minute (rpm). Taking into account a ring diameter of at least 1.2 meters and operation of RF communication in the V-band, this rotational movement causes a considerable Doppler effect due to the relative motion, which can lead to frequency shifts of up to 5 kHz.

The typical gap between rotor 102 and stator 103 is approximately 1 mm, which allows their relative rotation. However, this gap is limited by the axial displacement accuracy of the rotary transformer and the bearing tolerances. Reducing this clearance is critical to minimizing electromagnetic signal losses. Although the use of a special bearing is essential for the data transmission system, it can only reduce the gap to about 0.6 mm to 0.2 mm.

When using standard single square waveguides 101 in a rotary transformer, unwanted leakage occurs between the surfaces of the rotor 102 and stator 103 due to the gap in the structure. This leakage is directly proportional to the size of the gap 6, 106 relative to the wall dimensions of the waveguide 101 and is therefore undesirable as it increases the insertion loss.

According to the invention, therefore, a rib waveguide concept is used because it bundles the electromagnetic field mainly between the rib or ribs 105 and thus prevents the field in large extend from escaping through the gap 6, 106.

Three basic variants of the rib waveguide are proposed, namely a configuration with single ribs (SRWG)—see FIG. 1 and FIG. 2, with two ribs (DRWG)—see FIG. 3—and with four ribs (QRWG), with square ribs 105 and rectangular waveguides 101 being used in these embodiments.

As shown in FIGS. 5, 6, 7, 8 and 9, the invention is not limited to this, and other shapes may be used for both the ribs 105 and the waveguides 101.

The waveguide 101 should be terminated with a termination 112 made of absorbent material. This is important in order to prevent the propagation of multiple echoes through the waveguide 101, which is designed in a ring around the common axis of rotation.

The basic structure of the waveguide 101 was optimized by adapting the main dimensions as shown in Table 1.

According to the invention, the electromagnetic field is bundled between the ribs 105 so that almost no field leaks through the gap 6, 106. This shows that leakage is higher in the configuration with only one rib 105 in FIGS. 1 and 2 than in the embodiment with two ribs 105 in FIG. 3.

Table 1 shows calculated dimensions of the configurations according to the invention for the V-band (mm).

| | Single rib | Double rib | Quadruple rib |
|---|---|---|---|
| longest wall | 3.39 | 5.01 | 8.08 |
| shortest wall | 2.24 | 2.2 | 2.19 |
| Width of the rib | 0.2 | 1 | 0.83 |
| Space between the ribs | 0.65 | 0.75 | 0.7 |
| Separation of the edges | — | — | 2.84 |

For waveguide 101 with a single rib 105, “d” is the distance between the rib 105 and the opposite wall or within a two-part rib 105 in FIG. 2.

The feed into the waveguide 101 can be achieved by a combination of a T-branch and pyramidal horn transition, which width is matched to the width of the waveguide. This ensures feeding in the single mode and propagation by converting the main mode (TE10) into the desired mode of the waveguide 101.

In a preferred embodiment, shown in FIG. 4, a termination 112 is designed to absorb electromagnetic waves and minimize reflections. The termination 112 has the shape of two pyramids connected at the base and is made of absorbing material with regard to electromagnetic waves.

The basic geometry of the termination 112 or absorber is shown in FIG. 11. It comprises an absorbent wall 210, which ensures efficient absorption, and a matching pyramidal frustum 211, which reduces reflections. The same principle underlies other geometries that perform similar performance characteristics.

For example, the height of the pyramid can be 5 mm and the wall thickness of the absorber 4 mm.

Other waveguide profiles are possible, as shown in the figures. It is possible that shapes such as dog bone, butterfly or barbell designs may improve RF characteristics, including insertion loss or return loss.

The invention has been described above for transmission in the V-band. However, it is not limited to this. Transmissions in the E-band or in the Ka-band, in the frequency range from 27 to 40 GHz, can also be used with appropriate insulation and shielding.

The individual components for signal processing are cheaper in Ka-band than in V-band, but the system must be isolated in such a way that no external interference can occur. The V-band therefore offers the advantage that it does not require complex isolation.

### LIST OF REFERENCE SYMBOLS

1 gantry
2 Waveguide transmission system
3 X-ray tubes
4 X-ray detectors
5 patients
6 Gap
7 Transmitter
8 Receiver
9 Computer
101 Waveguide
102 Rotor
103 Stator
104 Gap
105 Rib
112 Termination
a Internal stator width
b Distance rotor to stator
c Width rib
d Distance stator to rib
e Distance ribs

We claim:

1. A data transmission system for transmitting data between two parts (102, 103) mounted rotatably relative to one another about a common axis, one of the parts (102) comprising at least one transmitter (7) and the other part (103) comprising a receiver (8), the data transmission system having a waveguide (101) which is divided in a longitudinal direction and has at least one rib (105) in cross-section.

2. The data transmission system according to claim 1, wherein the waveguide (101) is formed with a single rib (105) in the center of the waveguide (101) along a wide waveguide wall.

3. The data transmission system according to claim 1, wherein the waveguide (101) is formed with a plurality of ribs (105).

4. The data transmission system according to claim 1, wherein the waveguide (101) is provided on one side of the transmitter (7) with at least one termination (112) made of an absorbent material.

5. The data transmission system of claim 4, wherein the at least one termination (112) is pyramidal.

6. The data transmission system of claim 1, wherein a cross-sectional shape of the waveguide (101) and/or an excitation principle of the waveguide (101) are configured for a single mode wave.

7. The data transmission system of claim 1, wherein the waveguide (101) has a cross-section selected from the group consisting of round, oval, butterfly-shaped, T-shaped, double-T-shaped or angular cross-sections.

8. The data transmission system of claim 1, wherein a plurality of annular waveguides (101) arranged in parallel are provided.

9. The data transmission system of claim 8, wherein at least one of the annularly extending waveguides (101) is divided into a plurality of circular segments on which signals of different transmitters can be transmitted.

10. The data transmission system of claim 1, wherein several parts of the waveguide (101) are designed as a phased array.

11. The data transmission system of claim 1, wherein the waveguide (101) is laterally slotted.

12. Computer tomograph comprising a data transmission system for transmitting data between two parts (102, 103) mounted rotatably relative to one another about a common axis, one of the parts (102) comprising at least one transmitter (7) and the other part (103) comprising a receiver (8), the data transmission system having a waveguide (101) which is divided in a longitudinal direction and has at least one rib (105) in cross-section.

13. The computer tomograph of claim 12, further comprising wherein the waveguide is:
formed with a single rib in the center of the waveguide along a wide waveguide wall;
formed with a plurality of ribs; or
provided on one side of the transmitter with a termination made of an absorbent material.

14. The computer tomograph of claim 12, further comprising wherein at least one of:
a cross-sectional shape of the waveguide is configured for a single mode wave; or
the cross-sectional shape is selected from the group consisting of round, oval, butterfly-shaped, T-shaped, double-T-shaped or angular cross-sections.

15. The computer tomograph of claim 12, further comprising wherein a plurality of annular waveguides arranged in parallel are provided, and wherein at least one of the annularly extending waveguides is divided into a plurality of circular segments on which signals of different transmitters can be transmitted.

16. The computer tomograph of claim 12, wherein the waveguide comprises several parts designed as a phased array and/or lateral slots.

17. The data transmission system according to claim 2, wherein the waveguide is provided on one side of the transmitter with a termination made of an absorbent material.

18. The data transmission system according to claim 3, wherein the waveguide is provided on one side of the transmitter with a termination made of an absorbent material.

19. A data transmission system for transmitting data between two parts mounted rotatably relative to one another about a common axis, one of the parts comprising at least one transmitter and the other part comprising a receiver the data transmission system having a waveguide which is divided in a longitudinal direction and has at least one rib in cross-section, wherein the waveguide is provided on one side of the transmitter with at least one termination made of an absorbent material, further wherein the cross-section one of round, oval, butterfly-shaped, T-shaped, double-T-shaped or angular cross-sections.

20. The data transmission system of claim 19, further comprising wherein the at least one rib is:
a single rib in the center of the waveguide along a wide waveguide wall; or
two or more ribs.
